(19) United States
(12) Patent Application Publication
(10) Pub. No.: US 2024/0415127 A1
(43) Pub. Date: Dec. 19, 2024

(54) PLANT PRIMING COMPOSITIONS AND METHODS OF USE THEREOF

(71) Applicant: ZERO GRAVITY SOLUTIONS, INC., Boca Raton, FL (US)

(72) Inventors: Raveendran POTTATHIL, Boca Raton, FL (US); Kiran KHARAT, Boca Raton, FL (US)

(21) Appl. No.: 18/834,973

(22) PCT Filed: Jan. 31, 2023
(86) PCT No.: PCT/US2023/061671

§ 371 Date: Jul. 31, 2024

Related U.S. Application Data
(60) Provisional application No. 63/305,329, filed on Feb. 1, 2022.

(51) Int. Cl. A01N 59/20; A01P 21/00; C12Q 1/6895

(52) U.S. Cl. CPC A01N 59/20; A01P 21/00; C12Q 1/6895; C12Q 2600/13

(57) ABSTRACT

Provided herein are compositions and methods to improve plant resistance to abiotic and biotic stress, thereby improving crop yield. Provided herein are compositions including zinc, copper. Compositions described herein have plant priming activity where the plant's defenses against abiotic and biotic stress are boosted.

### BACKGROUND

Plants are constantly confronted by both abiotic and biotic stressors that seriously reduce their productivity. Plant responses to these stressors are complex and involve numerous physiological, molecular, and cellular adaptations. Priming is an adaptive strategy that improves the defensive capacity of plants. This phenomenon is marked by an enhanced activation of induced defense mechanisms. Stimuli from pathogens, beneficial microbes, or arthropods, as well as chemicals and abiotic cues, can trigger the establishment of priming by acting as warning signals. Upon stimulus perception, changes may occur in the plant at the physiological, transcriptional, metabolic, and epigenetic levels. This phase is called the priming phase. Upon subsequent challenge, the plant effectively mounts a faster and/or stronger defense response that defines the post-challenge primed state and results in increased resistance and/or stress tolerance. Priming can be durable and maintained throughout the plant's life cycle and can even be transmitted to subsequent generations, therefore representing a type of plant immunological memory.

Tomato (Solanum lycopersicum L.) is an important crop model system. The tomato genome possesses about 35,000 genes; a rich resource available to scientists (See for example, Ref. 1). The basic chromosome number of tomato is 2n=24, and wild forms range from diploids to hexaploids (See for example, Ref. 2). An elicitor triggers a hypersensitivity response in plants. Elicitors are very diverse molecules with wide chemical diversity, except that they all trigger the hypersensitivity response (See for example, Refs. 3-5). An elicitor's initial binding to a receptor in or on the surface of the host plant cell triggers the hypersensitivity response by inducing some chemical pathways. The plants' cell has receptors for elicitors. The specific nature of these receptors is unknown. The mechanism of binding of the elicitor to the receptor that triggers the hypersensitivity response has not been clearly understood. Presumably, a signal transduction mechanism is activated by elicitor-receptor binding. This signal transduction pathway might involve calcium ions, and it is similar to the signal transduction pathways shown to be involved in some hormonal responses (See for example, Refs. 5-7).

During ageing and plant degradation, endogenous elicitors can be produced; they include reactive oxygen species (ROS), oligosaccharide, and protein fragments, also substances generated inside the plant cell, such as hormones (e.g., jasmonic acid, salicylic acid), galacturonide, and alginate oligomer (See for example, Ref. 9). Another type of elicitor is exogenous substances unrelated to the composition of plants. These are anabolic products of the pathogen that trigger the defense responses of the plant; these may be constituents of the outer membrane, cell wall, or can be excretions (See for example, Ref. 10).

The treatment of plants with elicitors or pathogen attacks causes a cascade of defense reactions; these reactions include an accumulation of a range of plant-defensive secondary metabolites in intact plants (See for example, Refs. 10-11). The regulation of metabolic pathways by multigene families at transcriptional and translational levels leads to activation or inhibition of various signaling pathways. The pathway genes are involved in the production of anti-microbial compounds as well as signaling molecules. The induction of the metabolic pathway has led to the identification of a novel plant defense system for which various mechanisms have been proposed, including salicylic acid and anti-microbial mediated compounds (See for example, Refs. 1,2,11).

Evidence shows that a combination of abiotic and biotic stress can have a positive effect on plant performance by reducing the susceptibility to biotic stressors. Such an interaction between both types of stressors points to a crosstalk between their respective signaling pathways. This crosstalk may be synergistic and/or antagonistic and include the involvement of phytohormones, transcription factors, kinase cascades, and reactive oxygen species (ROS). In certain cases, such crosstalk can lead to a cross-tolerance and enhancement of a plant's resistance against pathogens or stress. Crop plants are subjected to multiple stressors during their lifespan that greatly reduce productivity and threaten global food security.

There remains the need for compositions and methods to prime plants to increase resistance and/or stress tolerance, and increase crop yield.

### BRIEF SUMMARY

In view of the foregoing, there is a need for formulations that improve crop production. The present disclosure addresses this need, and provides additional benefits as well.

In an aspect, provided herein are methods of priming a plant against biotic stress factors including treating the plant with a composition comprising zinc, copper, and acid, where the acid is selected from citric acid, sulfuric acid, oxalic acid, humic acid, fulvic acid, boric acid, acetic acid, and optionally ammonium sulfate, and a combination thereof, and where the ratio of copper to zinc is between 1:2 and 1:20.

A method of testing a composition for plant priming capabilities including exposing a plant to the composition, and assaying for one or more biomarkers, where the one or more biomarkers is selected from the group consisting of one or more biomarkers include Jasmonate acid Pathway, Solyc12g009220.2, auxin transporter-encoding genes, gibberellin, Solyc02g068680.1, Solyc01g095770.3, Abscisic acid signaling pathway genes, signal peptides (Solyc12g049170.2, Solyc12g049150.1, Solyc12g049070.1, Solyc09g075410.3, Solyc12g100110.1, Solyc12g100080.1, Solyc07g017570.2), Aromatic amino acid decarboxylase 1A (Solyc08g068680.3) and Fatty acyl-CoA reductase (Solyc06g074390.3), peroxidase gene (Solyc01g067870.3, Solyc11g007220.2, Solyc02g014300.2, Solyc02g082090.3, Solyc12g017870.2, Solyc01g009400.3, Solyc01g067860.3, Solyc05g055320.3), AP2/ERF (Solyc12g009240.1) and NAC (Solyc07g066330.3), WRKY23 (Solyc01g079260), and/or defense protein genes (Solyc07g009040.3, Solyc12g096920.1, Solyc07g009090.3, Solyc07g009030.3, Solyc07g009100.3, Solyc12g009240.1). In embodiments, the composition is suitable for priming if expression of the one or more biomarkers is increased compared to a control. In embodiments, the control is a plant that was not exposed to the composition. In embodiments, the control is a plant that was exposed to a control composition. In embodiments, the composition is suitable for priming if expression of the one or more biomarkers is increased by at least 10% compared to the control. In embodiments, the composition is suitable for priming if expression of the one or more biomarkers is increased by at least 20% compared to the control.

A method of priming a plant by inducing expression of one or more biomarkers, where the one or more biomarkers is selected from the group consisting of one or more biomarkers include Jasmonate acid Pathway, Solyc12g009220.2, auxin transporter-encoding genes, gibberellin, Solyc02g068680.1, Solyc01g095770.3, Abscisic acid signaling pathway genes, signal peptides (Solyc12g049170.2, Solyc12g049150.1, Solyc12g049070.1, Solyc09g075410.3, Solyc12g100110.1, Solyc12g100080.1, Solyc07g017570.2), Aromatic amino acid decarboxylase 1A (Solyc08g068680.3) and Fatty acyl-CoA reductase (Solyc06g074390.3), peroxidase gene (Solyc01g067870.3, Solyc11g007220.2, Solyc02g014300.2, Solyc02g082090.3, Solyc12g017870.2, Solyc01g009400.3, Solyc01g067860.3, Solyc05g055320.3), AP2/ERF (Solyc12g009240.1) and NAC (Solyc07g066330.3), WRKY23 (Solyc01g079260), and/or defense protein genes (Solyc07g009040.3, Solyc12g096920.1, Solyc07g009090.3, Solyc07g009030.3, Solyc07g009100.3, Solyc12g009240.1).

In an aspect, provided herein is a method of priming a plant by exposing the plant to one or more gene products selected from the group consisting of jasmonate acid Pathway, Solyc12g009220.2, auxin transporter-encoding genes, gibberellin, Solyc02g068680.1, Solyc01g095770.3, Abscisic acid signaling pathway genes, signal peptides (Solyc12g049170.2, Solyc12g049150.1, Solyc12g049070.1, Solyc09g075410.3, Solyc12g100110.1, Solyc12g100080.1, Solyc07g017570.2), Aromatic amino acid decarboxylase 1A (Solyc08g068680.3) and Fatty acyl-CoA reductase (Solyc06g074390.3), peroxidase gene (Solyc01g067870.3, Solyc11g007220.2, Solyc02g014300.2, Solyc02g082090.3, Solyc12g017870.2, Solyc01g009400.3, Solyc01g067860.3, Solyc05g055320.3), AP2/ERF (Solyc12g009240.1) and NAC (Solyc07g066330.3), WRKY23 (Solyc01g079260), and/or defense protein genes (Solyc07g009040.3, Solyc12g096920.1, Solyc07g009090.3, Solyc07g009030.3, Solyc07g009100.3, Solyc12g009240.1).

Provided herein is a plant priming composition comprising one or more gene products selected from the group consisting of Jasmonate acid Pathway, Solyc12g009220.2, auxin transporter-encoding genes, gibberellin, Solyc02g068680.1, Solyc01g095770.3, Abscisic acid signaling pathway genes, signal peptides (Solyc12g049170.2, Solyc12g049150.1, Solyc12g049070.1, Solyc09g075410.3, Solyc12g100110.1, Solyc12g100080.1, Solyc07g017570.2), Aromatic amino acid decarboxylase 1A (Solyc08g068680.3) and Fatty acyl-CoA reductase (Solyc06g074390.3), peroxidase gene (Solyc01g067870.3, Solyc11g007220.2, Solyc02g014300.2, Solyc02g082090.3, Solyc12g017870.2, Solyc01g009400.3, Solyc01g067860.3, Solyc05g055320.3), AP2/ERF (Solyc12g009240.1) and NAC (Solyc07g066330.3), WRKY23 (Solyc01g079260), and/or defense protein genes (Solyc07g009040.3, Solyc12g096920.1, Solyc07g009090.3, Solyc07g009030.3, Solyc07g009100.3, Solyc12g009240.1).

### BRIEF DESCRIPTION OF THE DRAWINGS

FIGS. 1A-1D demonstrate analysis of secondary metabolites of tomato seedlings by GCMS/MS.

FIGS. 2A-2C present volcano plot for all expressed genes in BamFX treated seeds-vs-untreated seeds.

FIGS. 3A-3C are graphs illustrating gene expression.

FIGS. 4A-4C present hierarchal clustering of top 25 genes expressed.

### DETAILED DESCRIPTION

### I. Definitions

The practice of the technology described herein will employ, unless indicated specifically to the contrary, conventional methods of chemistry, biochemistry, organic chemistry, molecular biology, microbiology, agriculture, and plant biology that are within the skill of the art, many of which are described below for the purpose of illustration. Examples of such techniques are available in the literature.

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art.

As used herein, the singular terms “a”, “an”, and “the” include the plural reference unless the context clearly indicates otherwise.

Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

As used herein, the terms “disease” or “condition” are used in accordance with its plain ordinary meaning and refer to a state of being or health status of a plant capable of being diagnosed and/or treated with compounds or methods provided herein. In embodiments, conditions include abiotic stress. In embodiments, conditions include biotic stress. Diseases include but are not limited to conditions caused by viruses, bacteria, fungus, insects, and combinations thereof.

As used herein, the term “abiotic stress” is used in accordance with its plain ordinary meaning and refers to the negative impact of non-living factors on the living organisms in a specific environment. Examples of abiotic stress in plants include drought, salinity, heat, cold, phosphate starvation, metal toxicity, and a combination thereof.

As used herein, the term “biotic stress” is used in accordance with its plain ordinary meaning and refers to living disturbances or the impact of living factors on the living organisms in a specific environment. Examples of biotic stress in plants include fungus, viral, bacterial, yeast, nematode, arachnid, or insect infection or infestations. Biotic stress may refer to infectious diseases that develop in harvested fruit that is caused by bacteria, fungi, or yeasts. Biotic stress may emerge from weeds among crops.

As used herein, the term “priming” or “plant priming” is used in accordance with its plain ordinary meaning and refers to a physiological process by which a plant prepares to more quickly or aggressively respond to future biotic or abiotic stress. The condition of readiness achieved by priming has been termed the “primed state.” Priming may be initiated in response to an environmental cue that reliably indicates an increased probability of encountering a biotic or abiotic stress, but a primed state may also persist as a residual effect following an initial exposure to the stress. For example, the classic pathogen-induced hypersensitive response is often induced with greater efficiency in plants that have previously experienced pathogen attack. In the context of long-lived plants such as trees, a primed state may persist across multiple growing seasons, a phenomenon commonly referred to in the ecological literature as “delayed induced resistance’. Because priming initiates a state of readiness that does not confer resistance per se but rather allows for accelerated induced resistance once an attack occurs, one presumed benefit of priming is that it does not impose the costs associated with full implementation of an induced defense response.

As used herein, “biomarker” refers to a measureable indicator of the physiological state of a plant or seed. The biomarker may be one or more of specific cells, molecules, metabolites, or genes, gene products, proteins, enzymes, or hormones. For example, the presence of a biomarker may indicate that a plant is responding to biotic or abiotic stress.

As used herein, the term “prevent” is used in accordance with its plain ordinary meaning and refers to a decrease in the occurrence of disease symptoms in a plant. The prevention may be complete (no detectable symptoms) or partial, such that fewer symptoms are observed than would likely occur absent treatment. Symptoms include but are not limited to vulnerability to disease, vulnerability to pests, lower growth size, lower crop yield, and decreased seed viability.

As used herein, the term “agriculture composition” and “horticulture composition” are used in accordance with its plain ordinary meaning and refer to a composition used with agriculture crops including but not limited to vegetables, fruit, nuts, grains, and cotton and with horticulture, including flowers, house plants, and the like.

As used herein, the term “copper (II) sulfate pentahydrate” refers to a compound with the following chemical formulation: CuSO₄·5H₂O or CuSO₄·5H₂O or CuH₁₀O₉S. It is alternatively known as “copper sulfate pentahydrate”, “Blue vitriol”, and “cupric sulfate pentahydrate.” The amount of copper in the total compound is 25%.

As used herein, the term “zinc sulfate monohydrate” refers to a compound with the following chemical formulation ZnSO₄·H₂. It is alternatively known a “zinc sulfate hydrate”, “white vitriol” and “goslarite.” The amount of zinc in the total compound is 36%.

As used herein, the term “copper sulfate pentahydrate” or “copper (II) sulfate pentahydrate” refers to a compound with the chemical formulation CuSO₄·5H₂O. Copper sulfate pentahydrate may also be known as “blue vitriol”, “bluestone”, “vitriol of copper”, or “Roman vitriol”. The amount of copper in the total compound is 25%.

As used herein, the term “citric acid” refers to a compound with the chemical formulation C₆H₈O₇. When part of a salt, the formula of the citrate anion may be written as C₆H₅O³⁻₇ or C₃H₅O(COO)³⁻.

As used herein, the term “sulfuric acid” refers to a compound with the chemical formulation H₂SO₄. Sulfuric acid may be referred to as “oil of vitriol”.

As used herein, the term “oxalic acid” refers to a compound with the chemical formulation C₂H₂O₄. Oxalic acid may occur as the dihydrate with the chemical formula oxalic acid occurs as the dihydrate with the formula C₂H₂O₄·2H₂O.

As used herein, the term “humic acid” refers to a class of compounds extracted as colloidal particles from soil into strong basic solutions, and precipitated from the basic solution by adjusting the pH to 1 with acid. Typically, the acid is hydrochloric acid.

As used herein, the term “fulvic acid” refers to a class of organic acids which are naturally occurring in soil organic matter. A fulvic acid may have the chemical formulation C₁₃₅H₁₈₂O₉₅N₅S₂.

As used herein, the term “boric acid” refers to a compound with the chemical formulation H₃BO₃, which may also be written as B(OH)₃. Boric acid may also be referred to as “hydrogen borate”, “boracic acid”, or “orthoboric acid”.

As used herein, the term “acetic acid” refers to a compound with the chemical formulation CH₃COOH, which may also be written as CH₃CO₂H, C₂H₄O₂, or HC₂H₃O₂. Acetic acid may also be referred to as “ethanoic acid”.

As used herein, the term “ammonium sulfate” refers to a compound with the chemical formulation (NH₄)₂SO₄.

As used herein, the term “iron sulfate heptahydrate” or “iron (II) sulfate heptahydrate” refers to a compound with the chemical formulation FeSO₄·7H₂O. Iron sulfate heptahydrate may also be referred to as iron (II) sulphate or ferrous sulfate. Other salts of iron (II) sulfate exist, and are denoted by the formula FeSO₄·xH₂O.

As used herein, the term “calcium lignin sulfate” refers to a compound with the chemical formulation C₂₀H₂₄CaO₁₀S₂. Calcium lignin sulfate may also be referred to as “calcium lignosulfonate” or “lignosulfonic acid, calcium salt”. Calcium lignin sulfate may be utilized as an encapsulating agent for compositions (i.e. BAM-dry formulation).

Compounds described herein may be further described by their physical form. For example, the physical form may be granulation or particle size. For example, copper (II) sulfate pentahydrate may be referred to as large (approximate particle size 8-25 mm), medium (approximate particle size 4-8 mm), small (approximate particle size 1-4 mm), Fine 20 (approximate particle size 20-40 mesh), Fine 30 (approximate particle size 30-100 mesh), which have crystal appearance, Fine 100 (approximate particle size 60-200 mesh), which has a powder appearance, or Fine 200 (approximate particle size 60-325 mesh), which has a fine powder appearance.

As used herein, the term “effective amount” is used in accordance with its plain ordinary meaning and refers to an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations.

As used herein, the term “therapeutically effective amount” is used in accordance with its plain ordinary meaning and refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the plant species and the area being treated. The dose administered to a plant, in the context of the present disclosure, should be sufficient to affect a beneficial therapeutic response in the plant over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular indication being treated. This will provide a therapeutic regimen that is commensurate with the severity of the plant's disease state.

As used herein, the term “administering” is used in accordance with its plain ordinary meaning and refers to application of a formulation for treatment of a plant or crop. In embodiments described herein, administering includes applying a formulation described herein to a plant part. For example, formulations described herein may be in a dry powder form that may be reconstituted in liquid. The liquid may then be applied as a foliar spray for applying to plant leaves, stems, or roots. Alternatively, seeds may be soaked in the reconstituted formulation for an amount of time. In other embodiments, formulations described herein may be in a wet or liquid formulation and applied as a foliar spray directly onto the plant or diluted and applied as a drench to the soil.

As used herein, the term “foliar spray” is used in accordance with its plain ordinary meaning and refers to a specific technique of applying a formulation to the leaves of a plant.

As used herein, the term “soil drench” is used in accordance with its plain ordinary meaning and refers to a specific technique of applying a diluted chemical pesticide, herbicide, fungicide, or even fertilizer to a particular plant or tree, or to a specific group of plants, rather than the entire garden or crop.

As used herein, the term “co-administer” is used in accordance with its plain ordinary meaning and refers to composition described herein is administered at the same time, just prior to, or just after the administration of one or more additional compounds, formulations, or treatments. The compounds provided herein can be administered alone or can be co-administered to the plant. Co-administration is meant to include simultaneous or sequential administration of the compounds individually or in combination (more than one compound). Thus, the preparations can also be combined, when desired, with other active substances.

As used herein, the term “cell” is used in accordance with its plain ordinary meaning and refers to a cell carrying out metabolic or other function sufficient to preserve or replicate its genomic DNA. In embodiments, cells include eukaryotic plant cells. In embodiments, cells include prokaryotic cells that include but are not limited to bacteria.

As used herein, the term “control” and “control experiment” are used in accordance with its plain ordinary meaning and refer to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of activity or effect in a plant in the absence of a compound as described herein (including embodiments and examples).

As used herein, the term “signaling pathway” is used in accordance with its plain ordinary meaning and refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

As used herein, the term “ROS” and “reactive oxygen species” are used in accordance with its plain ordinary meaning and refer to chemically reactive chemical species containing oxygen. Examples include peroxides, superoxide, hydroxyl radical, singlet oxygen, and alpha-oxygen. In a biological context, ROS are formed as a natural byproduct of the normal metabolism of oxygen and have important roles in cell signaling and homeostasis. However, during times of environmental stress (e.g., UV or heat exposure), ROS levels can increase dramatically. This may result in significant damage to cell structures. Cumulatively, this is known as oxidative stress. The production of ROS is strongly influenced by stress factor responses in plants, these factors that increase ROS production include drought, salinity, chilling, nutrient deficiency, metal toxicity and UV-B radiation. ROS may be generated by exogenous sources such as ionizing radiation.

As used herein, the term “phytohormone” or “plant hormone” are used in accordance with its plain ordinary meaning and refer to signal molecules produced within plants, that occur in extremely low concentrations. Plant hormones control all aspects of growth and development, from embryogenesis, the regulation of organ size, pathogen defense, stress tolerance and through to reproductive development. Unlike in animals (in which hormone production is restricted to specialized glands) each plant cell is capable of producing hormones

As used herein, the term “callose” is used in accordance with its plain ordinary meaning and refers to a polysaccharide in the form of beta-1,3-glucan with some beta-1,6-branches and it exists in the cell walls of a wide variety of higher plants. Callose is involved during a variety of processes in plant development and/or in response to multiple biotic and abiotic stresses.

As used herein, the terms “BRIX”, “degrees BRIX”, “BRIX content”, “° Bx “, and “°BRIX” are used in accordance with their plain ordinary meaning and refer to the sugar content of an aqueous solution. One degree Brix is 1 gram of sucrose in 100 grams of solution and represents the strength of the solution as percentage by mass. If the solution contains dissolved solids other than pure sucrose, then the ° Bx only approximates the dissolved solid content. The ° Bx is traditionally used in the wine, sugar, carbonated beverage, fruit juice, maple syrup and honey industries. Brix is used in the food industry for measuring the approximate amount of sugars in fruits, vegetables, juices, wine, soft drinks and in the starch and sugar manufacturing industry.

As used herein, the term “encapsulate” or “enclose” refers to surrounding something (i.e. a composition) on all sides, or confining something within a container. For example, BAM-FX or BAM-O may be enclosed within a capsule made of calcium lignosulfonate. Encapsulating may preserve active properties of the composition. Encapsulating may protect the composition from contaminants. Encapsulating may protect the surroundings from the composition.

As used herein, the term “gene product” is used in accordance with its plain ordinary meaning and refers to products of genes, for example RNA (e.g., messenger RNA (mRNA)) or protein. In embodiments, the gene product is an RNA. In embodiments, the gene product is an mRNA. In embodiments, the gene product is a protein.

### II. Compositions

In an aspect, provided herein are compositions including zinc, copper, and an acid, where the acid is selected from citric acid, sulfuric acid, oxalic acid, humic acid, fulvic acid, boric acid, acetic acid, and a combination thereof, and optionally ammonium sulfate, where the ratio of copper to zinc is between 1:2 and 1:20, and where the composition has plant priming activity.

In embodiments, provided herein are compositions including 2% copper and 7% water soluble zinc.

In an aspect, provided herein is a plant priming composition including one or more gene products selected from the group consisting of Jasmonate acid Pathway, Solyc12g009220.2, auxin transporter-encoding genes, gibberellin, Solyc02g068680.1, Solyc01g095770.3, Abscisic acid signaling pathway genes, signal peptides (Solyc12g049170.2, Solyc12g049150.1, Solyc12g049070.1, Solyc09g075410.3, Solyc12g100110.1, Solyc12g100080.1, Solyc07g017570.2), Aromatic amino acid decarboxylase 1A (Solyc08g068680.3) and Fatty acyl-CoA reductase (Solyc06g074390.3), peroxidase gene (Solyc01g067870.3, Solyc11g007220.2, Solyc02g014300.2, Solyc02g082090.3, Solyc12g017870.2, Solyc01g009400.3, Solyc01g067860.3, Solyc05g055320.3), AP2/ERF (Solyc12g009240.1) and NAC (Solyc07g066330.3), WRKY23 (Solyc01g079260), and/or defense protein genes (Solyc07g009040.3, Solyc12g096920.1, Solyc07g009090.3, Solyc07g009030.3, Solyc07g009100.3, Solyc12g009240.1).

In embodiments, the composition includes a gene product of the jasmonate acid Pathway. In embodiments, the composition includes a gene product of Solyc12g009220.2. In embodiments, the composition includes a gene product of an auxin transporter-encoding gene. In embodiments, the composition includes gibberellin. In embodiments, the composition includes a gene product of Solyc02g068680.1. In embodiments, the composition includes a gene product of Solyc01g095770.3. In embodiments, the composition includes a gene product of the abscisic acid signaling pathway. In embodiments, the composition includes a signal peptide encoded by Solyc12g049170.2. In embodiments, the composition includes a signal peptide encoded by Solyc12g049150.1. In embodiments, the composition includes a signal peptide encoded by Solyc12g049070.1. In embodiments, the composition includes a signal peptide encoded by Solyc09g075410.3. In embodiments, the composition includes a signal peptide encoded by Solyc12g100110.1. In embodiments, the composition includes a signal peptide encoded by Solyc12g100080.1. In embodiments, the composition includes a signal peptide encoded by Solyc07g017570.2. In embodiments, the composition includes gene Aromatic amino acid decarboxylase 1A (Solyc08g068680.3). In embodiments, the composition includes Fatty acyl-CoA reductase (Solyc06g074390.3). In embodiments, the composition includes peroxidase encoded by Solyc01g067870.3. In embodiments, the composition includes peroxidase encoded by Solyc11g007220.2. In embodiments, the composition includes peroxidase encoded by Solyc02g014300.2. In embodiments, the composition includes peroxidase encoded by Solyc02g082090.3. In embodiments, the composition includes peroxidase encoded by Solyc12g017870.2. In embodiments, the composition includes peroxidase encoded by Solyc01g009400.3. In embodiments, the composition includes peroxidase encoded by Solyc01g067860.3. In embodiments, the composition includes peroxidase encoded by Solyc05g055320.3. In embodiments, the composition includes AP2/ERF (such as encoded by Solyc12g009240.1). In embodiments, the composition includes NAC (such as encoded by Solyc07g066330.3). In embodiments, the composition includes WRKY23 (such as encoded by Solyc01g079260). In embodiments, the composition includes a defense protein selected from one or more proteins encoded by Solyc07g009040.3, Solyc12g096920.1, Solyc07g009090.3, Solyc07g009030.3, Solyc07g009100.3, or Solyc12g009240.

### Methods of Use

In an aspect, provided herein are methods of reducing cellular damage to a plant including treating the plant with a composition including zinc, copper, and acid, where the acid is selected from citric acid, sulfuric acid, oxalic acid, humic acid, fulvic acid, boric acid, acetic acid, and a combination thereof, and optionally ammonium sulfate, and where the ratio of copper to zinc is between 1:2 and 1:20.

In embodiments, provided herein are compositions including zinc and copper that induce resistance priming mechanisms in tomato seedlings. In embodiments, seeds treated with the composition exhibit differential gene expression in certain transcription factors and signal proteins. In embodiments, seeds treated with the composition exhibit differential gene expression in oxidative stress genes, defense protein and hormones. In embodiments, the compositions provided herein upregulate the stress related genes leading to development of disease resistance crop varieties in future.

In embodiments, cellular damage includes one or more of destructive protein modifications, mutagenic DNA strand breaks, purine oxidation, protein-DNA crosslinks, membrane leakage, cell lysis, and a combination thereof. In embodiments, the cellular damage is caused by reactive oxygen species. In embodiments, cellular damage includes destructive protein modifications. In embodiments, cellular damage includes mutagenic DNA strand breaks. In embodiments, cellular damage includes purine oxidation. In embodiments, cellular damage includes protein-DNA crosslinks. In embodiments, cellular damage includes membrane leakage. In embodiments, cellular damage includes cell lysis. In embodiments, cellular damage includes a combination of one or more of destructive protein modifications, mutagenic DNA strand breaks, purine oxidation, protein-DNA crosslinks, membrane leakage, and cell lysis. In embodiments, the cellular damage is caused by reactive oxygen species.

In embodiments, methods of reducing cellular damage to a plant include treating the plant with a composition as described herein and where the cellular damage is destructive protein modifications. In embodiments, methods of reducing cellular damage to a plant include treating the plant with a composition as described herein and the cellular damage is mutagenic DNA strand breaks. In embodiments, methods of reducing cellular damage to a plant include treating the plant with a composition as described herein and the cellular damage is purine oxidation. In embodiments, methods of reducing cellular damage to a plant include treating the plant with a composition as described herein and the cellular damage is protein-DNA crosslinks. In embodiments, methods of reducing cellular damage to a plant include treating the plant with a composition as described herein and the cellular damage is membrane leakage. In embodiments, methods of reducing cellular damage to a plant include treating the plant with a composition as described herein and the cellular damage is cell lysis. In embodiments, methods of reducing cellular damage to a plant include treating the plant with a composition as described herein and the cellular damage is a combination of one or more of destructive protein modifications, mutagenic DNA strand breaks, purine oxidations, protein-DNA cross links, membrane leakage, and cell lysis.

In embodiments, methods of reducing cellular damage to a plant include treating the plant with a composition as described herein. In embodiments, methods of reducing cellular damage to a plant including treating the plant with a composition including zinc sulfate monohydrate (ZnSO₄·H₂O), copper (II) sulfate pentahydrate (CuSO₄·5H₂O), and 10% (by weight) citric acid and where the copper to zinc ratio is 1:5.

In embodiments, methods of reducing cellular damage to a plant include induction of direct and/or indirect plant pathways for reducing cellular damage. In embodiments, the compositions described herein when applied to a plant surface including seeds, roots, leaves, and/or stems, prepares the plant for reducing cellular damage. Such preparation includes modulating gene expression, signaling pathways, and/or ion channels as required for reducing cellular damage. Examples of methods by which plants reduce cellular damage include reducing reactive oxygen species, increasing reactive species scavenging mechanisms, and production or increase of antioxidants.

In an aspect, provided herein are methods of priming a plant against abiotic stress factors including treating the plant with a composition including zinc and copper where the ratio of copper to zinc is between 1:2 and 1:20.

In an aspect, provided herein are methods of priming a plant against abiotic stress factors including treating the plant with a composition including one or more gene products selected from the group consisting of Jasmonate acid Pathway, Solyc12g009220.2, auxin transporter-encoding genes, gibberellin, Solyc02g068680.1, Solyc01g095770.3, Abscisic acid signaling pathway genes, signal peptides (Solyc12g049170.2, Solyc12g049150.1, Solyc12g049070.1, Solyc09g075410.3, Solyc12g100110.1, Solyc12g100080.1, Solyc07g017570.2), Aromatic amino acid decarboxylase 1A (Solyc08g068680.3) and Fatty acyl-CoA reductase (Solyc06g074390.3), peroxidase gene (Solyc01g067870.3, Solyc11g007220.2, Solyc02g014300.2, Solyc02g082090.3, Solyc12g017870.2, Solyc01g009400.3, Solyc01g067860.3, Solyc05g055320.3), AP2/ERF (Solyc12g009240.1) and NAC (Solyc07g066330.3), WRKY23 (Solyc01g079260), and/or defense protein genes (Solyc07g009040.3, Solyc12g096920.1, Solyc07g009090.3, Solyc07g009030.3, Solyc07g009100.3, Solyc12g009240.1).

In embodiments, provided herein are methods of priming a plant against abiotic stress factors including drought, salinity, heat, cold, phosphate starvation, metal toxicity, and a combination thereof. In embodiments, the abiotic stress factor is drought salinity. In embodiments, the abiotic stress factor is heat. In embodiments, the abiotic stress factor is cold. In embodiments, the abiotic stress factor is phosphate starvation. In embodiments, the abiotic stress factor is metal toxicity. In embodiments, the abiotic stress factor is a combination of one or more of drought, salinity, heat, cold, phosphate starvation, and metal toxicity.

In embodiments, methods of priming a plant against abiotic stress factors include treating the plant with a composition as described herein. In embodiments, methods of priming a plant against abiotic stress factors include treating the plant with a composition including zinc sulfate monohydrate (ZnSO₄·H₂O), copper (II) sulfate pentahydrate (CuSO₄·5H₂O), and 10% (by weight) citric acid and where the copper to zinc ratio is 1:5.

In embodiments, methods of priming against abiotic stress include induction of direct and/or indirect plant defenses. In embodiments, the compositions described herein when applied to a plant surface including seeds, roots, leaves, and/or stems, prepares the plant for defense against an abiotic stress. Such preparation includes modulating gene expression, signaling pathways, and/or ion channels as required for the particular abiotic stress.

In an aspect, provided herein are methods of promoting growth of a plant including treating the plant with a composition including zinc and copper where the ratio of copper to zinc is between 1:2 and 1:20.

In an aspect, provided herein are methods of promoting growth of a plant including treating the plant with a composition including one or more gene products selected from the group consisting of Jasmonate acid Pathway, Solyc12g009220.2, auxin transporter-encoding genes, gibberellin, Solyc02g068680.1, Solyc01g095770.3, Abscisic acid signaling pathway genes, signal peptides (Solyc12g049170.2, Solyc12g049150.1, Solyc12g049070.1, Solyc09g075410.3, Solyc12g100110.1, Solyc12g100080.1, Solyc07g017570.2), Aromatic amino acid decarboxylase 1A (Solyc08g068680.3) and Fatty acyl-CoA reductase (Solyc06g074390.3), peroxidase gene (Solyc01g067870.3, Solyc11g007220.2, Solyc02g014300.2, Solyc02g082090.3, Solyc12g017870.2, Solyc01g009400.3, Solyc01g067860.3, Solyc05g055320.3), AP2/ERF (Solyc12g009240.1) and NAC (Solyc07g066330.3), WRKY23 (Solyc01g079260), and/or defense protein genes (Solyc07g009040.3, Solyc12g096920.1, Solyc07g009090.3, Solyc07g009030.3, Solyc07g009100.3, Solyc12g009240.1).

In embodiments, methods of promoting growth of a plant includes induction of direct and/or indirect plant pathways for plant. In embodiments, the compositions described herein when applied to a plant surface including seeds, roots, leaves, and/or stems, improves and/or accelerates plant growth. Methods by which plant growth is improved or accelerated include modulating gene expression, signaling pathways, and/or ion channels as required for increase in yield, size, and/or weight of the plant, fruit, seed, nut, and/or flower.

In embodiments, promoting growth of a plant includes increasing crop yield, increasing plant height, increasing size of fruit, increasing size of vegetable, or nut weight, vegetable weight, or flower quantity, and a combination thereof. In embodiments, promoting growth of a plant includes increasing crop yield. In embodiments, promoting growth of a plant includes increasing plant height. In embodiments, promoting growth of a plant includes increasing size of fruit. In embodiments, promoting growth of a plant includes increasing size of vegetable. In embodiments, promoting growth of a plant includes nut weight. In embodiments, promoting growth of a plant includes vegetable weight. In embodiments, promoting growth of a plant includes flower quantity.

In an aspect, provided herein are methods of priming a plant against biotic stress factors including treating the plant with a composition comprising zinc, copper, and acid, where the acid is selected from citric acid, sulfuric acid, oxalic acid, humic acid, fulvic acid, boric acid, acetic acid, and a combination thereof, and optionally ammonium sulfate, and where the ratio of copper to zinc is between 1:2 and 1:20.

In embodiments, priming a plant against biotic stress factors includes treating the plant with a composition as described herein. In embodiments, priming a plant against biotic stress factors includes treating the plant with a composition including zinc sulfate monohydrate (ZnSO₄·H₂O), copper (II) sulfate pentahydrate (CuSO₄·5H₂O), and 10% (by weight) citric acid and where the copper to zinc ratio is 1:5.

In embodiments, priming a plant against biotic stress factors includes treating the plant with a composition treating the plant with a composition including one or more gene products selected from the group consisting of Jasmonate acid Pathway, Solyc12g009220.2, auxin transporter-encoding genes, gibberellin, Solyc02g068680.1, Solyc01g095770.3, Abscisic acid signaling pathway genes, signal peptides (Solyc12g049170.2, Solyc12g049150.1, Solyc12g049070.1, Solyc09g075410.3, Solyc12g100110.1, Solyc12g100080.1, Solyc07g017570.2), Aromatic amino acid decarboxylase 1A (Solyc08g068680.3) and Fatty acyl-CoA reductase (Solyc06g074390.3), peroxidase gene (Solyc01g067870.3, Solyc11g007220.2, Solyc02g014300.2, Solyc02g082090.3, Solyc12g017870.2, Solyc01g009400.3, Solyc01g067860.3, Solyc05g055320.3), AP2/ERF (Solyc12g009240.1) and NAC (Solyc07g066330.3), WRKY23 (Solyc01g079260), and/or defense protein genes (Solyc07g009040.3, Solyc12g096920.1, Solyc07g009090.3, Solyc07g009030.3, Solyc07g009100.3, Solyc12g009240.1).

In embodiments, the plant is further treated with a composition comprising zinc, copper, and acid, where the acid is selected from citric acid, sulfuric acid, oxalic acid, humic acid, fulvic acid, boric acid, acetic acid, and a combination thereof, and optionally ammonium sulfate, and where the ratio of copper to zinc is between 1:2 and 1:20.

In embodiments, priming against biotic stress includes induction of direct and/or indirect plant defenses. In embodiments, the compositions described herein when applied to a plant surface including seeds, roots, leaves, and/or stems, prepares the plant for defense against a biotic stress. Such preparation includes modulating gene expression, signaling pathways, and/or ion channels as required for the particular biotic stress.

In embodiments, priming includes induction of priming pathways or expression of biomarkers indicative of priming. In embodiments, priming includes induction of priming pathways. In embodiments, priming includes production of biomarkers indicative of priming. In embodiments, the biomarkers include carboxylic acids. In embodiments, biomarkers include protein biomarkers. In embodiments, the protein biomarkers are biomarkers involved in anti-oxidant protective pathways. In embodiments, the protein biomarkers are transcription factors. In embodiments, the protein biomarkers are epigenetic markers. In embodiments, the biomarkers are chemical biomarkers. In embodiments, the chemical biomarkers are plant metabolites. In embodiments, the biomarkers are gene biomarkers.

In embodiments, the biomarkers include abscisic acid (ABA), salicylic acid (SA), jasmonic acid (JA), or ethylene (ET). In embodiments, the biomarker is abscisic acid (ABA). In embodiments, the biomarker is salicylic acid (SA). In embodiments, the biomarker is jasmonic acid (JA). In embodiments, the biomarker is ethylene (ET).

In embodiments, the biomarkers include Jasmonate acid Pathway, Solyc12g009220.2, auxin transporter-encoding genes, gibberellin, Solyc02g068680.1, Solyc01g095770.3, Abscisic acid signaling pathway genes, signal peptides (Solyc12g049170.2, Solyc12g049150.1, Solyc12g049070.1, Solyc09g075410.3, Solyc12g100110.1, Solyc12g100080.1, Solyc07g017570.2), Aromatic amino acid decarboxylase 1A (Solyc08g068680.3) and Fatty acyl-CoA reductase (Solyc06g074390.3), peroxidase gene (Solyc01g067870.3, Solyc11g007220.2, Solyc02g014300.2, Solyc02g082090.3, Solyc12g017870.2, Solyc01g009400.3, Solyc01g067860.3, Solyc05g055320.3), AP2/ERF (Solyc12g009240.1) and NAC (Solyc07g066330.3), WRKY23 (Solyc01g079260), and/or defense protein genes (Solyc07g009040.3, Solyc12g096920.1, Solyc07g009090.3, Solyc07g009030.3, Solyc07g009100.3, Solyc12g009240.1).

In embodiments, the biomarkers include a combination of two or more biomarkers provided herein.

In embodiments, the methods provided herein include treating the seeds of a plant with any of the compositions provided herein, including embodiments thereof. In embodiments, treating the seeds includes soaking the seeds in a solution including the composition.

In embodiments, a dry formulation of BAM-FX is produced. In embodiments, the dry formulation is combined with water to make a liquid formulation of BAM-FX. In embodiments, a stock or concentrate of BAM-FX is made with 400 grams of dry powder BAM-FX mixed in 1 liter of water. This stock solution may be further diluted.

In an aspect is provided a method of testing a composition for plant priming capabilities including exposing a plant to the composition, and assaying for one or more biomarkers, where the one or more biomarkers is selected from the group consisting of one or more biomarkers include Jasmonate acid Pathway, Solyc12g009220.2, auxin transporter-encoding genes, gibberellin, Solyc02g068680.1, Solyc01g095770.3, Abscisic acid signaling pathway genes, signal peptides (Solyc12g049170.2, Solyc12g049150.1, Solyc12g049070.1, Solyc09g075410.3, Solyc12g100110.1, Solyc12g100080.1, Solyc07g017570.2), Aromatic amino acid decarboxylase 1A (Solyc08g068680.3) and Fatty acyl-CoA reductase (Solyc06g074390.3), peroxidase gene (Solyc01g067870.3, Solyc11g007220.2, Solyc02g014300.2, Solyc02g082090.3, Solyc12g017870.2, Solyc01g009400.3, Solyc01g067860.3, Solyc05g055320.3), AP2/ERF (Solyc12g009240.1) and NAC (Solyc07g066330.3), WRKY23 (Solyc01g079260), and/or defense protein genes (Solyc07g009040.3, Solyc12g096920.1, Solyc07g009090.3, Solyc07g009030.3, Solyc07g009100.3, Solyc12g009240.1). In embodiments, the composition is suitable for priming if expression of the one or more biomarkers is increased compared to a control. In embodiments, the control is a plant that was not exposed to the composition. In embodiments, the control is a plant that was exposed to a control composition.

In embodiments, the composition is suitable for priming if expression of the one or more biomarkers is increased by at least 5% compared to the control. In embodiments, the composition is suitable for priming if expression of the one or more biomarkers is increased by at least 10% compared to the control. In embodiments, the composition is suitable for priming if expression of the one or more biomarkers is increased by at least 15% compared to the control. In embodiments, the composition is suitable for priming if expression of the one or more biomarkers is increased by at least 20% compared to the control. In embodiments, the composition is suitable for priming if expression of the one or more biomarkers is increased by at least 25% compared to the control. In embodiments, the composition is suitable for priming if expression of the one or more biomarkers is increased by at least 30% compared to the control. In embodiments, the composition is suitable for priming if expression of the one or more biomarkers is increased by at least 40% compared to the control. In embodiments, the composition is suitable for priming if expression of the one or more biomarkers is increased by at least 50% compared to the control.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein

### EXAMPLES

### Example 1: Tomato Seeds Germination in the Presence of Bam-FX

Tomato (Solanum lycopersicum L.) is an important crop that possesses about 35,000 genes. The treatment of plants with elicitors or pathogen attacks causes a cascade of defense reactions. The regulation of metabolic pathways by multigene families at transcriptional and translational levels leads to activation or inhibition of various signaling pathways. We investigated tomato responses to a solution containing Zn and Cu elicitors (BAM FX, nanoparticles of Copper (20 g/L) and Zinc (68 g/L)) and report the results of comparative transcriptome analysis of tomato seeds treated with Zn and Cu elicitors. The seeds were treated with optimal concentrations of Bam-FX solutions and subjected to cold methanolic extraction methods to obtain the secondary metabolites produced within them at different time intervals post-Bam-FX treatment. The metabolite mixture was analyzed using gas chromatography-mass spectrometry (GCMS). In transcriptome sequencing, GO and KEGG analzyes revealed that the majority of the DEGs in BamFx treated tomato was associated with primary and secondary metabolism, plant hormone signal transduction, TF regulation, transport, and responses to stimuli.

Elicitors induces protein expression of enzymes for the detoxification and phosphate degradation, membrane transports, transcription factors and signal transduction. The proteins from chloroplast, plasma membrane and cell wall are repressed by elicitors. We investigated tomato responses to the BamFX solution containing Zn and Cu elicitors and report the results of comparative transcriptome analysis of tomato seeds treated with Zn and Cu elicitors. The goals were to (i) construct a tomato seedling transcriptome; (ii) compare and analzye the transcripts in control and Zn and Cu elicitor-treated plants, and (iii) gain insight into stress tolerance and pathogen resistance induced by Cu and Zn in tomatoes. This study presents the transcriptome of tomato leaves responding to Zn and Cu elicitors and provides a genetic resource that can be used for crop improvement.

Germination of tomato seeds was observed in the presence of BamFX dilutions. Table 1 describes the effect of the BamFX 1:500 dilution (30 min) on the germination of the tomato seeds. The germination rate was 60% in tomato seeds after 48 h and increased to 94% after 72 h. When the seeds soaking time was increased up to 60 min in BamFX 1:500, the germination rate increased up to 68% after 48 h. (Table 1). When seeds were treated with BamFX1:1000 for 30 min, 70% of the seeds germinated after 48 h, increasing to 96% after 72 h. (Table 1).

TABLE 1
Germination percentage of Tomato seeds treated
with BamFX vs Untreated control seeds.

| | | Germination | Germination |
|---|---|---|---|
| BamFx | Duration of | percentage | percentage |
| dilution | exposure | after 48 h | after 72 h |
| BamFX 1:500 | 30 min | 60% ± 2.4% | 94% ± 1.5% |
| BamFX 1:500 | 60 min | 68% ± 2.5% | 96% ± 1.5% |
| BamFX 1:1000 | 30 min | 70% ± 2.5% | 96% ± 1.5% |
| BamFX 1:1000 | 60 min | 70% ± 2.5% | 96% ± 1.5% |
| Untreated | — | 42% ± 2.5% | 64% ± 1.5%\| |
| control | | | |

### Secondary Metabolite Analysis Using GCMS.

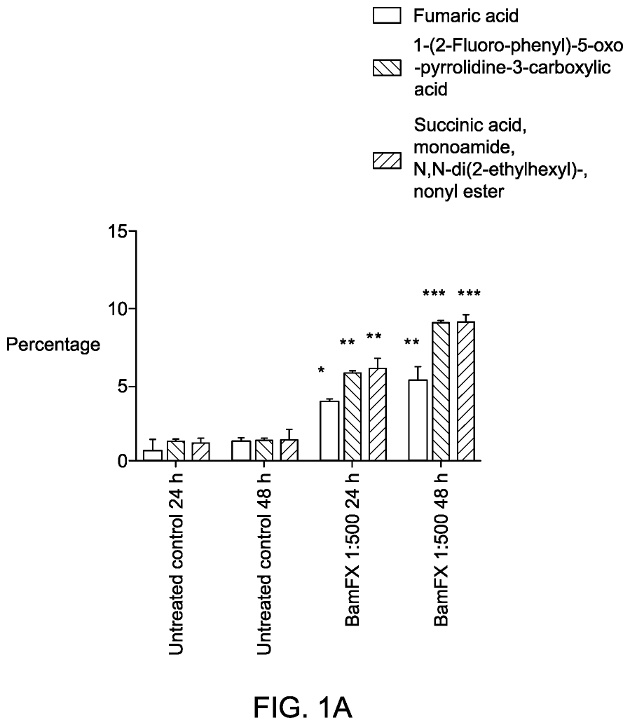

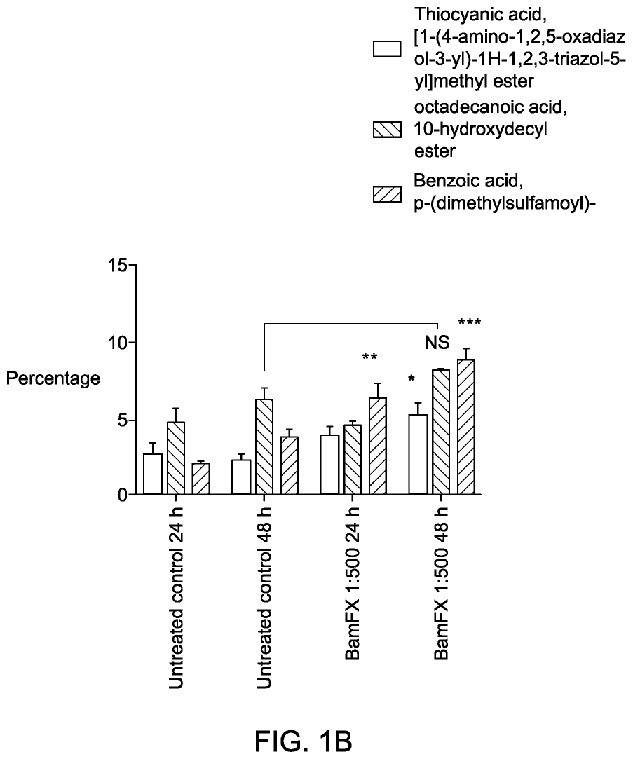

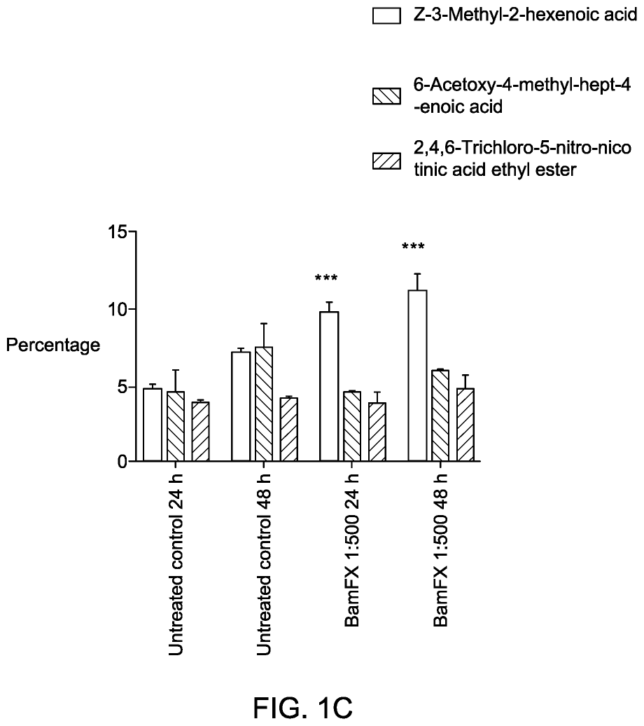

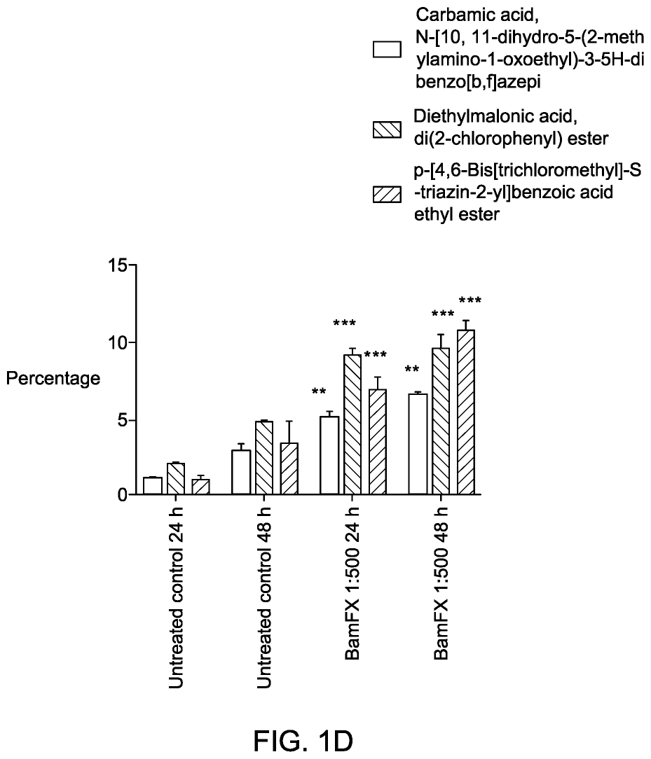

We used GCMS/MS for the analysis of the secondary metabolites from the tomato seeds treated with BamFX and untreated control. The secondary metabolites found in the BamFX treated tomato seedlings-Esters of Fumaric acid, Succinic acid, thiocyanic acid, octadecanoic acid, benzoic acid, hexenoic acid, heptanoic acid, Nicotinic acids, carbamic acid and Diethylmalonic acid. Fumaric acid, 1-(2-Fluoro-phenyl)-5-oxo-pyrrolidine-3-carboxylic acid (2-chloro-phenyl)-amide, Succinic acid, monoamide, N,N-di(2-ethylhexyl)-, nonyl ester, Thiocyanic acid, [1-(4-amino-1,2,5-oxadiazol-3-yl)-1H-1,2,3-triazol-5-yl]methyl ester, octadecanoic acid, 10-hydroxydecyl ester, Benzoic acid, p-(dimethylsulfamoyl)-, Carbamic acid, N-[10,11-dihydro-5-(2-methylamino-1-oxoethyl)-3-5Hdibenzo[b,f]azepi, Diethylmalonic acid, di(2-chlorophenyl) ester and p-[4,6-Bis[trichloromethyl]-S-triazin-2-yl]benzoic acid ethyl ester were found induced in the BamFX 1:500 treated seeds after 24 h of growth. (FIG. 1). Z-3-Methyl-2-hexenoic acid and 6-Acetoxy-4-methyl-hept-4-enoic acid were found decreased in the BamFX1:500 treated seeds than untreated control seeds. FIG. 1 shows analysis of secondary metabolites of tomato seedlings by GCMS/MS. The data are presented for untreated control 24 h-Untreated Tomato seeds incubated in sterile petri dish for 24 h; Untreated control 48 h-Untreated Tomato seeds incubated in sterile petri dish for 24 h; BamFX 1:500 24 h-Tomato seeds treated with BamFX 1:500 for 30 mins and incubated in sterile petri dish for 24 h; BamFX 1:500 48 h-Tomato seeds treated with BamFX 1:500 for 30 mins and incubated in sterile petridish for 48 h. Data are expressed as the secondary metabolites in seeds, where significance refers to the differences between BamFX treated and control untreated seeds (n=3; ****P<0.0001, ***P<0.001, **P<0.01, *P<0.05, ns=not significant). Error bars indicate SD.

### RNA-Seq Data Analysis

To explore differences in the molecular mechanisms of the defense between BamFX (elicitor treated) and untreated control tomato seedlings, we used Illumina sequencing technology to analyze the transcriptome profiles of the seedlings. A total of 23,558,528 raw reads were obtained. Approximately 22,954,544 clean reads with >95% Q30 bases (those with a base quality greater than 30) were selected as high-quality reads for further analysis (Table 1). The high-quality reads were mapped to the reference tomato transcript sequences, resulting in the mapping of approximately 96% of the nucleotides. Mapping revealed that transcripts of 18395, 18610, and 18229 genes were detected in the BamFX 1:500 and BamFX 1:1000 treated and untreated control seedlings, respectively.

### Functional Annotation and Classification of DEGs

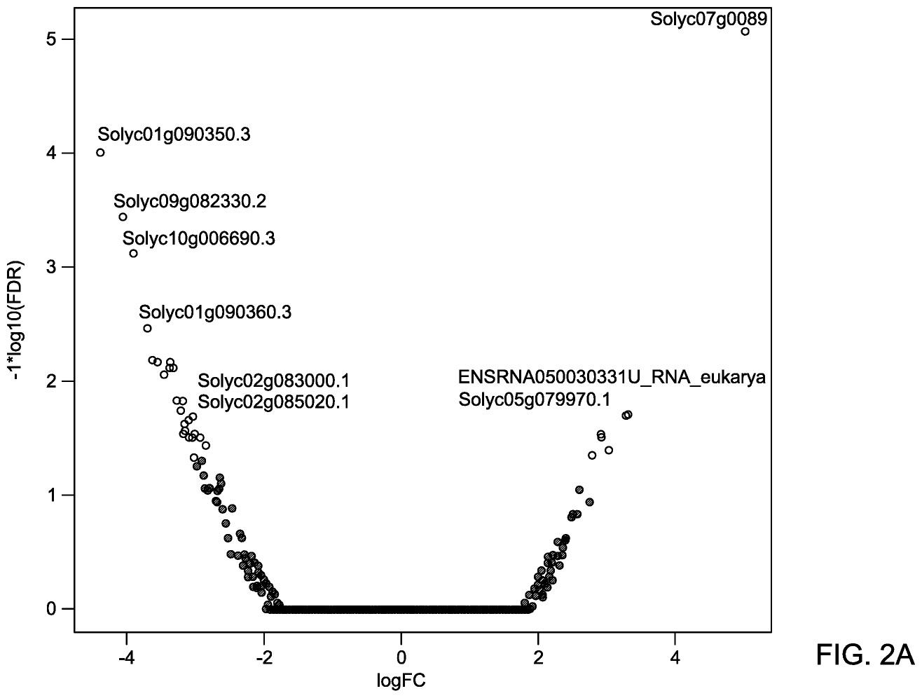

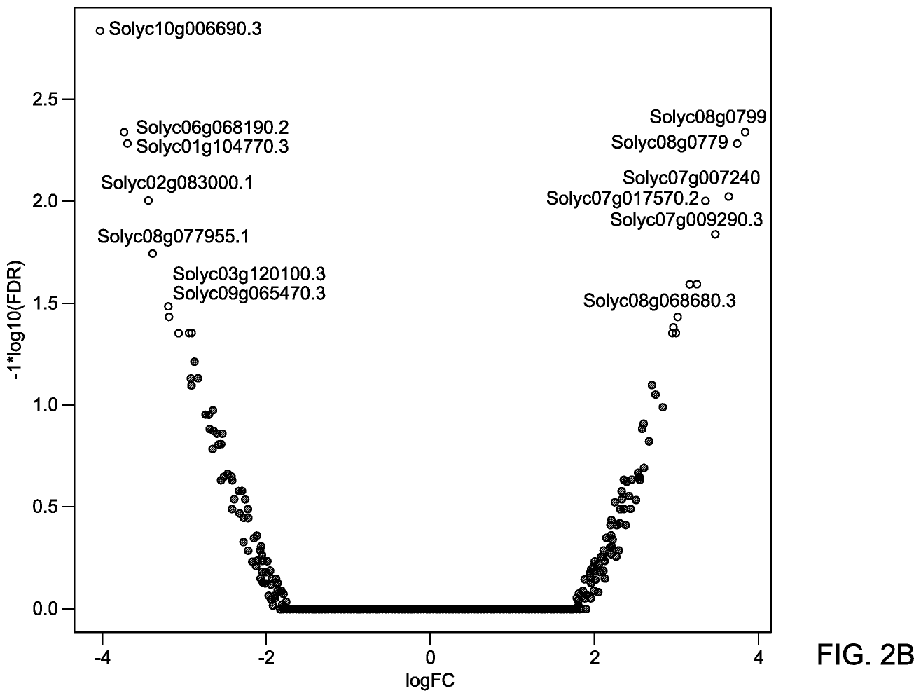

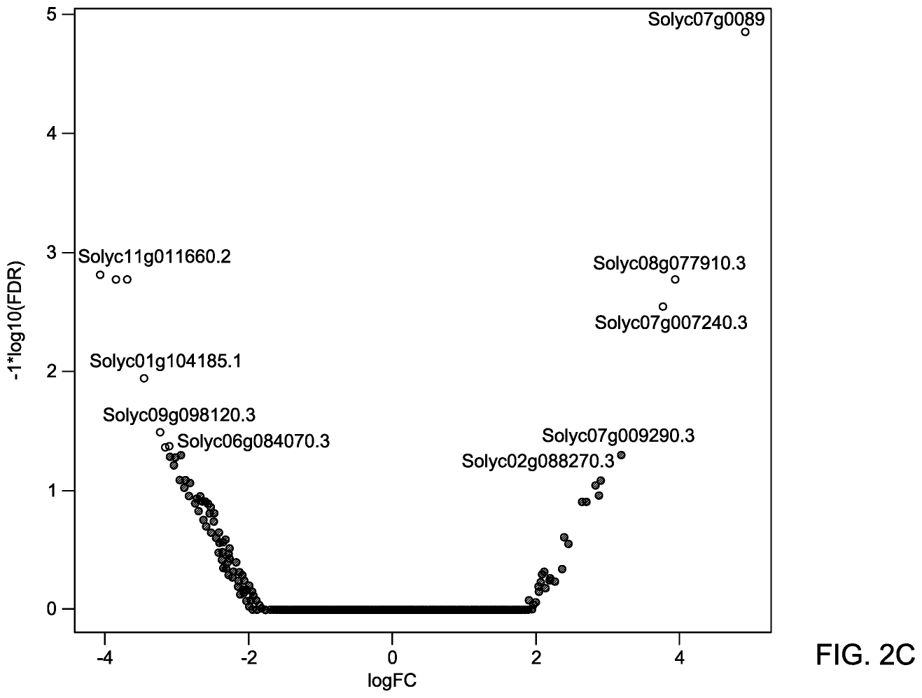

To identify the DEGs between the control (untreated seedlings) and BamFX-treated seedlings, we employed a general chi-squared test with false discovery rate (FDR) correction and a p-value of 0.05 using DEseq6 software to identify two-fold upregulated and two-fold down-regulated genes. In total 2016 genes, significantly DEGs were detected between the control and the treatment samples, with 1142 upregulated genes and 874 downregulated genes being detected in the BamFX samples (FIG. 2).

FIG. 2 shows volcano plots for all expressed genes in BamFX treated seeds-Vs-Untreated seeds. 2A) Tomato seeds treated with BamFx1:500 for 30 min. 2B) Tomato seeds treated with BamFX1:500 for 60 min. 2C) Tomato seeds treated with BamFx 1:1000 for 30 min.

In search of the possible functions of the Differentially expressed genes, local alignment search by BLAST for non-redundant proteins (NR), nucleotide sequences (NT), Clusters of Orthologous Groups (COG), UniProt, gene ontology (GO), and Kyoto Encyclopaedia of Genes and Genomes (KEGG) databases were performed.

### GO Enrichment Analysis of Differentially Expressed Genes.

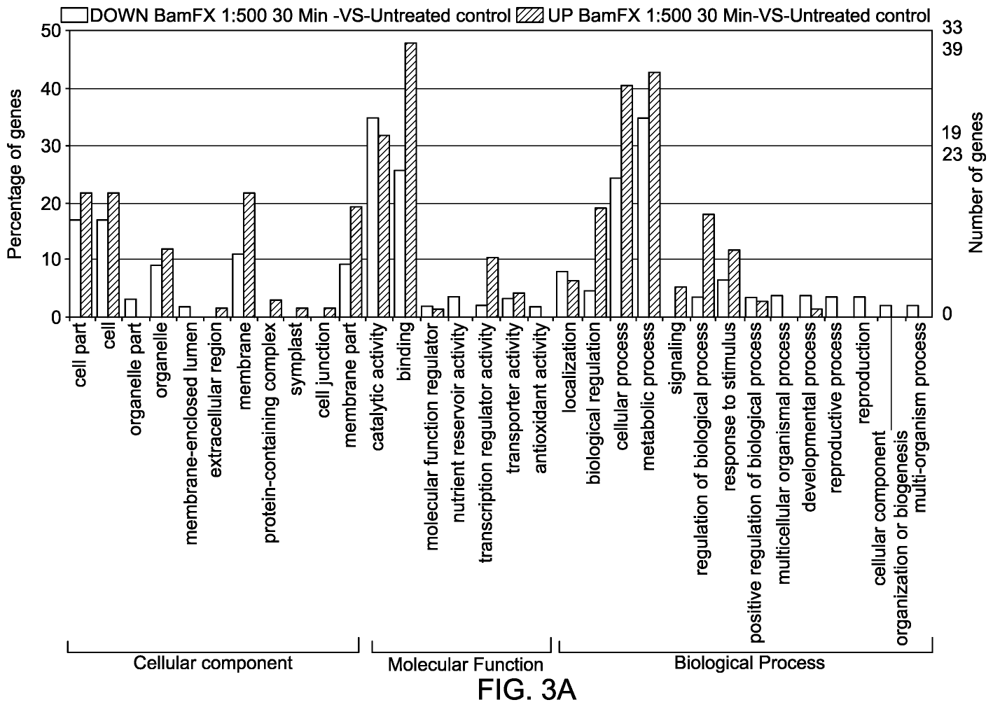

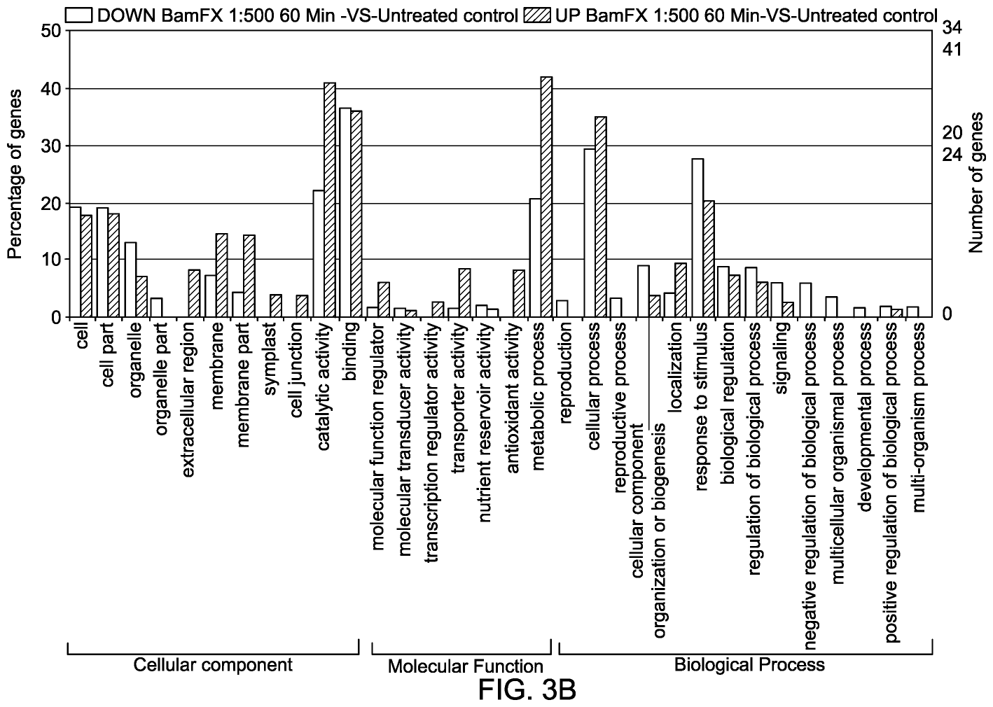

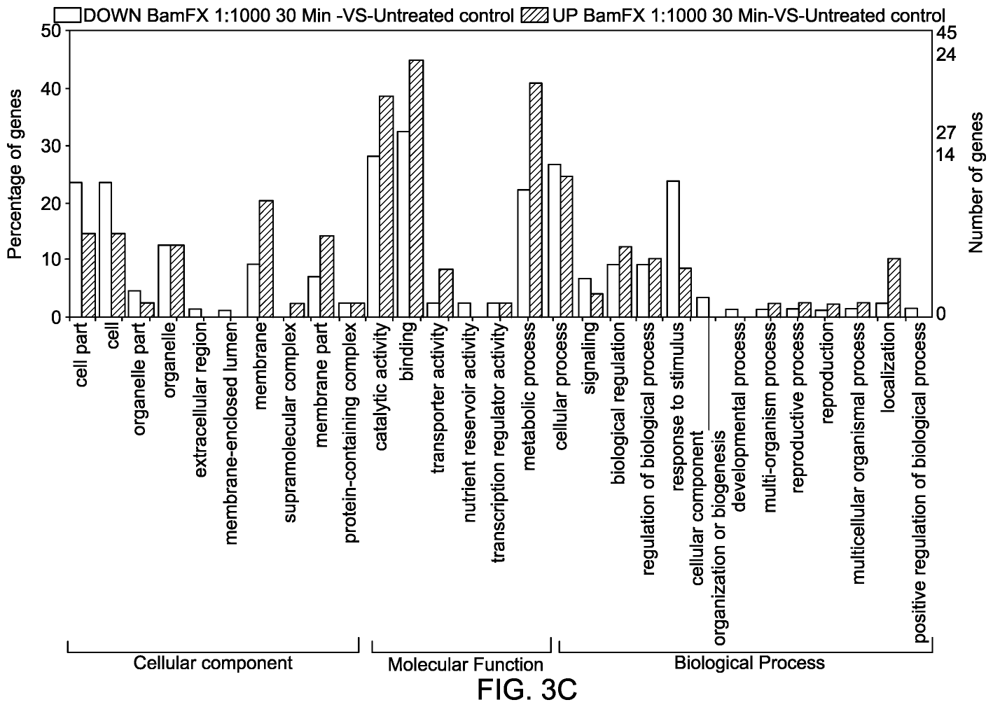

Based on the functions of each DEG, a GO enrichment analysis was performed. All the DEGs were grouped into more than 33 functional groups distributed into three main categories: cellular components, molecular functions, and biological processes (FIG. 3). The GO functions were significantly enriched in the BamFX-treated seedlings. The ‘organelle’, ‘cell part’, and ‘membrane terms’ from the cellular components category were significantly enriched. The ‘cellular processes’, ‘response to stimulus’, metabolic processes, and ‘biological regulation’ from the biological processes category were significantly enriched. Whereas from the molecular functions category, ‘catalytic activity’ and ‘protein binding’ were significantly enriched. Also, several DEGs were classified into two functional subclasses involved with transcription regulator activity and transporter activity. Thus, the majority of the identified DEGs were responsible for fundamental processes associated with biological regulation and metabolism (FIG. 3).

### KEGG Enrichment Analysis of DEGs

To group, the biological functions of the DEGs, a KEGG pathway enrichment analysis was performed. All the DEGs were analyzed by KEGG pathways. Most of the DEGs were protein processing in the endoplasmic reticulum and plant hormone signal transduction along with the photosynthesis proteins, biosynthesis of amino acids, mitogen-activated protein kinase (MAPK) signaling pathway, and carbon metabolism.

### Analysis of DEGs Between BamFx-Treated and Untreated Seedlings in the Plant Hormone Signal Pathways

In BamFX-treated (1:500 for 30 min) seeds, changes in genes associated with the Jasmonate acid pathway were identified. The significant upregulation of genes in the Jasmonate acid signaling pathways is associated with pathogen infection, plant hormones, and wounding. Solyc12g009220.2 was upregulated in BamFx-treated plantlets. Most genes associated with the regulation of diverse hormones were differentially expressed between BamFx-treated and untreated seedlings. The transcriptome analysis showed that the expression of genes associated with protein ubiquitination changed significantly. We speculated that these hormone signaling pathways might be involved in differences between BamFx-treated and untreated tomato seedlings.

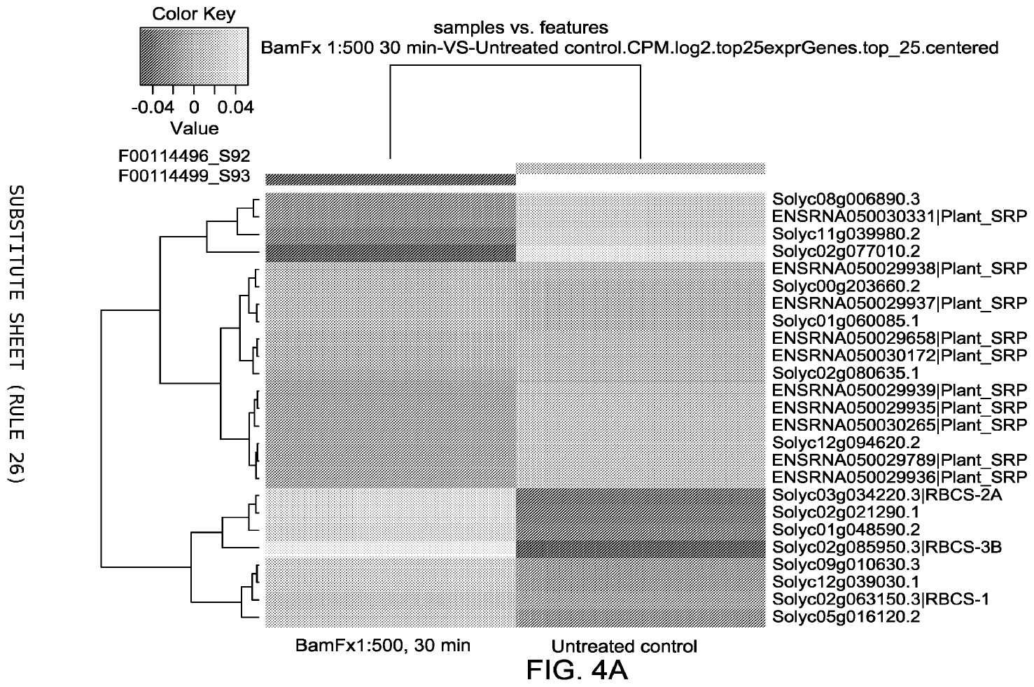

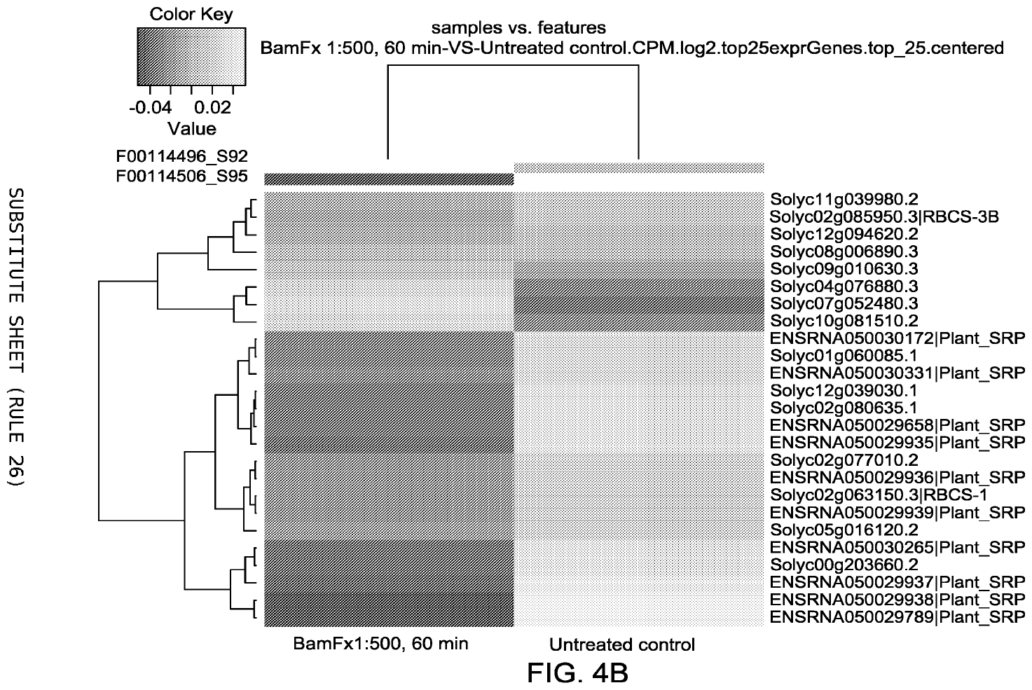

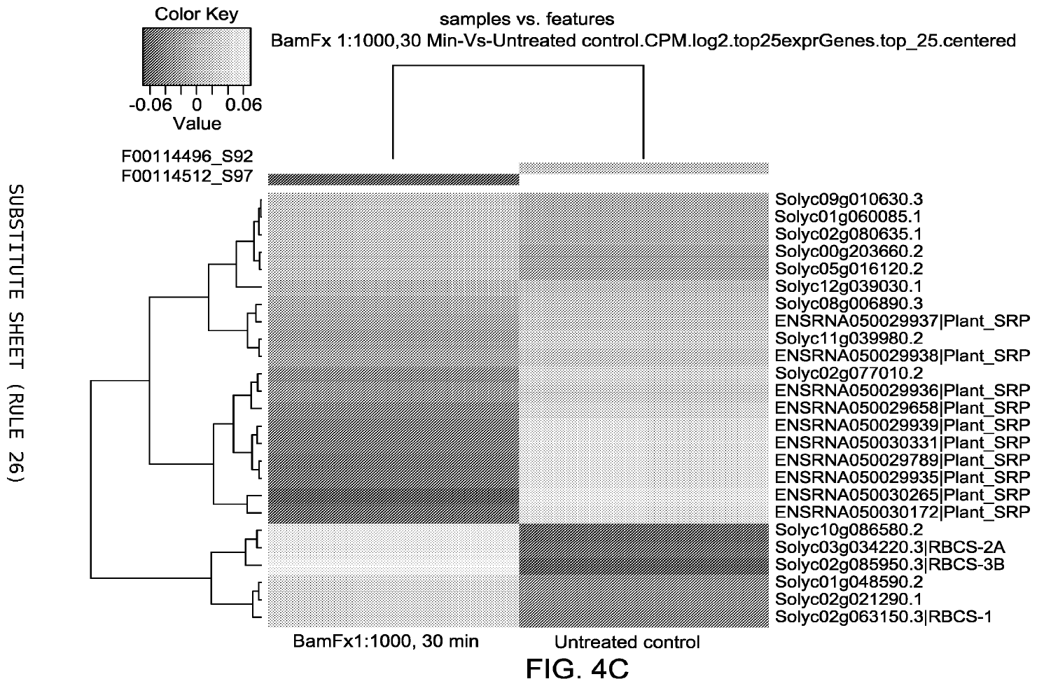

The transcript levels of most auxin transporter-encoding genes changed significantly in the BamFX treated seedlings (e.g., Solyc01g007010.3, a RING-type E3 ubiquitin transferase). The gibberellin is important to enhance cell elongation and induce cell division. The gene Solyc07g061720.3 for Gibberellin 2-oxidase was upregulated in the BamFx-treated seedlings. The Phorbol-ester/DAG-type domain containing protein (Solyc02g068680.1) associated with the intracellular signaling gene was upregulated in the BamFx-treated seeds. Also, we identified six upregulated genes involved in the protein kinase activity signaling pathways in the BamFX-treated seedlings. Solyc01g095770.3 (involved in ion channel activity) was upregulated (FIG. 4). FIG. 4 shows Hierarchical clustering of top 25 expressers where 4A shows Tomato seeds treated with BamFx1:500 for 30 min. 4B shows Tomato seeds treated with BamFX1:500 for 60 min. 4C shows Tomato seeds treated with BamFx 1:1000 for 30 min.

The time-dependent effect of the BamFX (1:500 for 60 min) was found to be regulating many signal transduction pathways. Abscisic acid signaling pathway genes (Solyc09g015380.1) were upregulated in BamFX-treated (1:500 for 60 min) plants. Many signal peptides (Solyc12g049170.2, Solyc12g049150.1, Solyc12g049070.1, Solyc09g075410.3, Solyc12g100110.1, Solyc12g100080.1, Solyc07g017570.2) were upregulated in BamFX-treated (1:500 for 60 min) plants (FIG. 4).

Protein kinases expression was elevated in the BamFX-treated (1:500 for 60 min) plants. Solyc12g036325.1, Solyc04g079710.3, and Solyc03g119340.3 were upregulated at the lower concentrations of the BamFX (1:1000 for 30 min). Auxin signaling pathway genes (Solyc08g021820.3, Solyc02g082450.3) and an inorganic phosphate transporter (Solyc03g005530.1) were upregulated (FIG. 4).

### Carboxylic Acid Pathways

Aromatic amino acid decarboxylase 1A (Solyc08g068680.3) and Fatty acyl-CoA reductase (Solyc06g074390.3) were upregulated in the BamFX-treated (1:500 for 60 min) plants.

Aromatic amino acid decarboxylase 1A (Solyc08g068680.3) and Cytochrome b561 domain-containing protein (Solyc07g048070.3) were upregulated in BamFX (1:1000 for 30 min) (FIG. 4).

### Analysis of Oxidative Stress Genes Differentially Expressed Between BamFx-Treated and Untreated Seedlings

The reactive oxygen species are produced during photosynthesis and respiration. The low production of ROS is under strict regulation of the plant cells. Environmental stress can cause an increase in ROS contents. The antioxidant system in plants can remove excess ROS and maintain normal metabolism. In the present study, the significant expression of several candidate genes associated with ROS scavengings, such as Prephenate/arogenate dehydrogenase (Solyc09g011870.2), Fe2OG dioxygenase (Solyc12g006370.2), and L-ascorbate oxidase (Solyc04g054690.3), supported the differential regulation of oxidative stress mechanisms between BamFx-treated and untreated tomato seedlings (FIGS. 3 and 4).

When tomato seeds were exposed to BamFx 1:500 for 60 min, peroxidase gene expression was elevated (Solyc01g067870.3, Solyc11g007220.2, Solyc02g014300.2, Solyc02g082090.3, Solyc12g017870.2, Solyc01g009400.3, Solyc01g067860.3, Solyc05g055320.3). This expression profile was not found in seedlings exposed to BamFX 1:500 for 30 min. Other enzymes such as Fe20G dioxygenase Solyc09g089780.3 were also expressed and upregulated in seedlings treated with BamFx 1:500 for 60 min. (FIGS. 3 and 4)

### TFs in the BamFx-Treated Tomato Seedlings

Members of the complex family of WRKY TFs are associated with the transcription regulation associated with the plant immune system. In this study, the expression of many WRKY TFs was upregulated very significantly in BamFx-treated seeds. TFs are involved in gene regulation strictly connected with responses to stress; therefore, the genetic manipulation of TFs is highly desirable. In the present analysis, four TFs were differentially expressed in the BamFX-treated seedlings (FIG. 4).

WRKY family members are also directly involved in abiotic stress signaling and tolerance. For example, WRKY23 (Solyc01g079260) responds to auxin regulation, and WRKY70 participates in the defense response to fungus attacks. The present results supported the broad functions of this TF gene family in tomatoes. AP2/ERF (Solyc12g009240.1) and NAC (Solyc07g066330.3) were upregulated in the BamFX treated (1:500 for 60 min) seedlings (FIG. 4).

### Defense Proteins in BamFX-Treated Seedlings.

The expression of defense proteins was upregulated in BamFX-treated (1:500 for 30 min) plants. Solyc12g096920.1, Solyc04g007780.3, and Solyc07g009090.3 were up-regulated in the BamFx-treated seeds. In BamFX-treated (1:500 for 60 min) tomato seedlings, the number of genes upregulated was more than with BamFX-treated (1:500 for 30 min) tomato seedlings; and the defense-related gene expression was found upregulated (Solyc07g009040.3, Solyc12g096920.1, Solyc07g009090.3, Solyc07g009030.3, Solyc07g009100.3, Solyc12g009240.1) (FIG. 4).

### Discussion

The results analysis of the enriched GO terms revealed that the DEGs were determined to be associated with the enzymatic regulation of metabolism, stress response and signal transduction. BamFX altered the transcription of genes regulating major mechanisms which includes the rearrangement of cell cycle, cell division and regular metabolic pattern. The BamFX affected the antioxidant defense system by upregulating expression of genes with active regulation of the oxidative stress in plants. The activation of PAMP leading to PTI is the primary level of response that is induced by plant microbial interaction, whereas the secondary level of response is the induction of ETI by recognition of the effectors secreted within the plant cells by intracellular immune receptors (See for example, Refs. 15-17).

The MAPK pathway is associated with various mechanisms in plant cells such as the biotic and abiotic stresses, regulation of hormones, cell division and differentiation along with the responses to pathogens and abiotic stresses (See for example Refs. 16-19). In the present study, the biological functions of the DEGs were identified by applying KEGG analysis. The majority of the genes were upregulated in the BamFx-treated seedlings in association with the phytohormones signal transduction and MAPK pathway. The expression of a RING type E3 ubiquitin transferase, an auxin transporter-encoding gene was observed as elevated in the BamFX-treated seedlings. The time-dependent effect of the BamFX was recorded to be regulating many signal transduction pathways. The Phorbol-ester/DAG-type domain-containing, which are associated with the intracellular signaling gene, was upregulated in the BamFx-treated plants. Abscisic acid signaling pathway genes and Protein kinases expression were found as elevated at the lower concentrations of the BamFX. The Auxin signaling pathway genes and an inorganic phosphate transporter along with Aromatic amino acid decarboxylase 1A and Fatty acyl-CoA reductase were upregulated. The increased transcript level of the important genes associated with ROS scavenging, such as Prephenate/arogenate dehydrogenase, Fe20G dioxygenase and L-ascorbate oxidase supported the differential regulation of oxidative stress mechanisms.

In conclusion, BamFX induced resistance priming mechanisms in tomato seedlings. The differential gene expression in BamFX treated seedlings revealed the induction of transcription factors and upgraded signal proteins. The oxidative stress genes, defense protein and hormones were found upregulated in the BamFX treated seedlings. The study reports upregulation of the stress related genes leading to development of disease resistance crop varieties in future.

### Methods

Seeds Treatment with BamFX Dilutions and the Seed Germination Rate.

Tomato seeds were taken in a Petri dish. The seeds were soaked in BamFX 1:500 and 1:1000 dilutions for 30 min and 60 min.

After 30 min or 60 min soaking in the BamFX dilutions, seeds were removed from the plate and kept in a sterile Petri dish containing wet tissue paper. The growth of the seeds was observed and recorded. The seeds grown after 48 h sent the RNA extraction method for the sequencing.

### Plant Material for RNA-Seq

Seeds of tomato were planted and grown in plastic pots and grown at room temperature. Fifty pots (five seedlings per pot) were used in this experiment.

RNA Extraction, cDNA Library Construction, and Illumina Deep Sequencing

Trizol reagent (Thermo, USA) used for the preparation of total RNA from tomato seedlings.

The mRNA was purified and used for the library construction with the Truseq™ RNA Sample Prep Kit (Illumina, San Diego, CA, USA) following the manufacturer's instructions. The six samples were sequenced on an Illumina HiSeq™ 2000 (Illumina). Each sample yielded more than 12 Gb of data. Sequencing was completed by the Neuberg Biology Lab, Ahmedabad, India.

### Read Trimming and Optimization

The sequencing adapters were trimmed for each set of sequencing reads, using SeqPrep (github.com/jstjohn/SeqPrep), and then low-quality bases (Solexa/Illumina quality score <25) of the 3′ ends were trimmed using in-house Perl scripts.

The quality reads were used for the mapping analysis against the reference genome sequences (ftp.solgenomics.net/tomato_genome/annotation/ITAG2.3_release/) using Tophat. The Cufflink was used to assemble all mapped reads. The assembled results and original genome annotations were merged and used for further annotation and differential expression analysis.

### Mapping Reads to the Reference Genome and Annotated Genes

Open reading frames (ORFs in all transcripts were predicted using Trinity (trinityrnaseq.sourceforge.net/analysis/extract_proteins_from_trinity_transcripts.html). Sequence similarity Blast searches of these transcripts were conducted against the tomato genome reference, the NCBI NR protein database (www.ncbi.nlm.nih.gov/), the Gene Ontology (GO) database (www.geneontology.org/), the Search Tool for the Retrieval of Interacting Genes (STING) database (string-db.org/), and the Kyoto Encyclopedia of Genes and Genomes (KEGG) database (www.genome.jp/kegg/). GO terms for tomato transcripts were obtained using Blast2GO (v. 2.3.5) (www.blast2go.org/) with default parameters. COG terms were obtained using Blastx 2.2.24+ in STRING 9.0. Metabolic pathways were analzyed by using Blastx/Blastp 2.2.24+ in KEGG (www.genome.jp/kegg/genes.html).

### Differential Expression Analysis

The Tophat (tophat.cbcb.umd.edu/) and Cufflinks (cufflinks.cbcb.umd.edu/) programs provide FPKM (Fragments Per Kilobase of exon model per Million mapped fragments) values within a 95% confidence interval. Differential expression was analyzed and calculated according to the count values of each transcript in the two libraries using edgeR (the Empirical Analysis of Digital Gene Expression in R) software. “FDR<0.05” and “|log 2 fold-change (log 2FC)|≥1” were used as the thresholds for judging significant differences in transcript expression. Transcripts with |log 2FC|<0.25 were assumed to have no change in expression levels.

### Statistics

Data are reported as mean±S.D. All experiments were done at least three times, and three or more independent observations were made on each occasion. Statistically significant values were compared using one-way analysis of variance (ANOVA) and p-values less than 0.05 were considered statistically significant.

### REFERENCES

1. The tomato genome sequence provides insights into fleshy fruit evolution. Nature 485, (2012).
2. Gupta, S. et al. Transcriptome profiling of cytokinin and auxin regulation in tomato root. Journal of Experimental Botany 64, (2013).
3. Finkelstein, R. R., Gampala, S. S. L. & Rock, C. D. Abscisic Acid Signaling in Seeds and Seedlings. The Plant Cell 14, (2002).
4. Raghavendra, A. S., Gonugunta, V. K., Christmann, A. & Grill, E. ABA perception and signaling. Trends in Plant Science 15, (2010).
5. Chinnusamy, V., Gong, Z. & Zhu, J.-K. Abscisic acid-mediated Epigenetic Processes in Plant Development and Stress Responses. Journal of Integrative Plant Biology 50, (2008).
6. Cutler, S. R., Rodriguez, P. L., Finkelstein, R. R. & Abrams, S. R. Abscisic Acid: Emergence of a Core Signaling Network. Annual Review of Plant Biology 61, (2010).
7. Fujita, Y., Fujita, M., Shinozaki, K. & Yamaguchi-Shinozaki, K. ABA-mediated transcriptional regulation in response to osmotic stress in plants. Journal of Plant Research 124, (2011).
8. Wang, R.-S. et al. Common and unique elements of the ABA-regulated transcriptome of Arabidopsis guard cells. BMC Genomics 12, (2011).
9. Kumar, S., Kaur, G. & Nayyar, H. Exogenous Application of Abscisic Acid Improves Cold Tolerance in Chickpea (Cicer arietinum L.). Journal of Agronomy and Crop Science (2008) doi: 10.1111/j.1439-037X.2008.00335.x.
10. Kumar, S., Kaushal, N., Nayyar, H. & Gaur, P. Abscisic acid induces heat tolerance in chickpea (Cicerarietinum L.) seedlings by the facilitated accumulation of osmoprotectants. Acta Physiologiae Plantarum 34, (2012).
11. Grant, M. R. & Jones, J. D. G. Hormone (Dis) harmony Moulds Plant Health and Disease. Science 324, (2009).
12. Mauch-Mani, B. & Mauch, F. The role of abscisic acid in plant-pathogen interactions. Current Opinion in Plant Biology 8, (2005).
13. Audenaert, K., De Meyer, G. B. & Höfte, M. M. Abscisic Acid Determines Basal Susceptibility of Tomato to Botrytis cinerea and Suppresses Salicylic acid-dependent Signaling Mechanisms. Plant Physiology 128, (2002).
14. Song, W., Ma, X., Tan, H. & Zhou, J. Abscisic acid enhances resistance to Alternaria solani in tomato seedlings. Plant Physiology and Biochemistry 49, (2011).
15. Abramovitch, R. B., Anderson, J. C. & Martin, G. B. Bacterial elicitation and evasion of plant innate immunity. Nature Reviews Molecular Cell Biology 7, (2006).
16. (Kazuya Ichimura et al.), M. G. et al. Mitogen-activated protein kinase cascades in plants: a new nomenclature. Trends in Plant Science 7, (2002).
17. Gallego-Giraldo, L. et al. Elicitors and defense gene induction in plants with altered lignin compositions. New Phytologist 219, (2018).
18. Miedes, E., Vanholme, R., Boerjan, W. & Molina, A. The role of the secondary cell wall in plant resistance to pathogens. Frontiers in Plant Science 5, (2014).
19. Zhao, Q. & Dixon, R. A. Altering the Cell Wall and Its Impact on Plant Disease: From Forage to Bioenergy. Annual Review of Phytopathology 52, (2014).

What is claimed is:

1. A method of priming a tomato plant against abiotic stress factors comprising treating the plant with a composition comprising zinc, copper and acid wherein the ratio of copper to zinc is between 1:2 and 1:20; and measuring upregulation of one or more biomarkers selected from oxidative stress genes, defense protein genes and hormone genes.

2. A method of priming a tomato plant against biotic stress factors comprising treating the plant with a composition comprising zinc and copper wherein the ratio of copper to zinc is between 1:2 and 1:20; and measuring upregulation of upregulation of oxidative stress genes, defense protein genes and hormone genes.

3. The method of claim 1 or 2, wherein treating the plant comprises treating a seed of the plant with the composition.

4. The method of claim 3, wherein the seed is soaked in a solution comprising the composition.

5. The method of claim 2 or 3, wherein the ratio of copper to zinc is 1:10.

6. The method of claim 2 or 3, wherein the zinc is zinc sulfate monohydrate (ZnSO₄·H₂O).

7. The method of claim 6, wherein the zinc sulfate monohydrate (ZnSO₄·H₂O) has a zinc content of 36%.

8. The method of claim 1 or 2, wherein following treatment the plant increases production of one or more plant priming biomarkers.

9. The method of claim 8, wherein the one or more biomarkers include Jasmonate acid Pathway, Solyc12g009220.2, auxin transporter-encoding genes, gibberellin, Solyc02g068680.1, Solyc01g095770.3, Abscisic acid signaling pathway genes, signal peptides (Solyc12g049170.2, Solyc12g049150.1, Solyc12g049070.1, Solyc09g075410.3, Solyc12g100110.1, Solyc12g100080.1, Solyc07g017570.2), Aromatic amino acid decarboxylase 1A (Solyc08g068680.3) and Fatty acyl-CoA reductase (Solyc06g074390.3), peroxidase gene (Solyc01g067870.3, Solyc11g007220.2, Solyc02g014300.2, Solyc02g082090.3, Solyc12g017870.2, Solyc01g009400.3, Solyc01g067860.3, Solyc05g055320.3), AP2/ERF (Solyc12g009240.1) and NAC (Solyc07g066330.3), WRKY23 (Solyc01g079260), and/or defense protein genes (Solyc07g009040.3, Solyc12g096920.1, Solyc07g009090.3, Solyc07g009030.3, Solyc07g009100.3, Solyc12g009240.1).

10. The method of any of the preceding claims, wherein the copper is copper (II) sulfate pentahydrate (CuSO₄·5H₂O).

11. The method of any of the preceding claims, wherein the copper (II) sulfate pentahydrate (CuSO₄·5H₂O) has a copper content of 25%.

12. A method of testing a composition for plant priming capabilities comprising:
a. exposing a plant to the composition, and
b. assaying for one or more biomarkers, wherein the one or more biomarkers is selected from the group consisting of one or more biomarkers include Jasmonate acid Pathway, Solyc12g009220.2, auxin transporter-encoding genes, gibberellin, Solyc02g068680.1, Solyc01g095770.3, Abscisic acid signaling pathway genes, signal peptides (Solyc12g049170.2, Solyc12g049150.1, Solyc12g049070.1, Solyc09g075410.3, Solyc12g100110.1, Solyc12g100080.1, Solyc07g017570.2), Aromatic amino acid decarboxylase 1A (Solyc08g068680.3) and Fatty acyl-CoA reductase (Solyc06g074390.3), peroxidase gene (Solyc01g067870.3, Solyc11g007220.2, Solyc02g014300.2, Solyc02g082090.3, Solyc12g017870.2, Solyc01g009400.3, Solyc01g067860.3, Solyc05g055320.3), AP2/ERF (Solyc12g009240.1) and NAC (Solyc07g066330.3), WRKY23 (Solyc01g079260), and/or defense protein genes (Solyc07g009040.3, Solyc12g096920.1, Solyc07g009090.3, Solyc07g009030.3, Solyc07g009100.3, Solyc12g009240.1).

13. A method of priming a plant by inducing expression of one or more biomarkers, comprising contacting the plant with a composition; where the one or more biomarkers is selected from the group consisting of a Jasmonate acid Pathway protein, Solyc12g009220.2, auxin transporter-encoding genes, gibberellin, Solyc02g068680.1, Solyc01g095770.3, Abscisic acid signaling pathway genes, signal peptides (Solyc12g049170.2, Solyc12g049150.1, Solyc12g049070.1, Solyc09g075410.3, Solyc12g100110.1, Solyc12g100080.1, Solyc07g017570.2), Aromatic amino acid decarboxylase 1A (Solyc08g068680.3) and Fatty acyl-CoA reductase (Solyc06g074390.3), peroxidase gene (Solyc01g067870.3, Solyc11g007220.2, Solyc02g014300.2, Solyc02g082090.3, Solyc12g017870.2, Solyc01g009400.3, Solyc01g067860.3, Solyc05g055320.3), AP2/ERF (Solyc12g009240.1) and NAC (Solyc07g066330.3), WRKY23 (Solyc01g079260), and/or defense protein genes (Solyc07g009040.3, Solyc12g096920.1, Solyc07g009090.3, Solyc07g009030.3, Solyc07g009100.3, Solyc12g009240.1).

14. A method of priming a plant comprising exposing the plant to one or more gene products selected from the group consisting of a Jasmonate acid Pathway, Solyc12g009220.2, auxin transporter-encoding genes, gibberellin, Solyc02g068680.1, Solyc01g095770.3, Abscisic acid signaling pathway genes, signal peptides (Solyc12g049170.2, Solyc12g049150.1, Solyc12g049070.1, Solyc09g075410.3, Solyc12g100110.1, Solyc12g100080.1, Solyc07g017570.2), Aromatic amino acid decarboxylase 1A (Solyc08g068680.3) and Fatty acyl-CoA reductase (Solyc06g074390.3), peroxidase gene (Solyc01g067870.3, Solyc11g007220.2, Solyc02g014300.2, Solyc02g082090.3, Solyc12g017870.2, Solyc01g009400.3, Solyc01g067860.3, Solyc05g055320.3), AP2/ERF (Solyc12g009240.1) and NAC (Solyc07g066330.3), WRKY23 (Solyc01g079260), and/or defense protein genes (Solyc07g009040.3, Solyc12g096920.1, Solyc07g009090.3, Solyc07g009030.3, Solyc07g009100.3, Solyc12g009240.1).

15. A plant priming composition comprising one or more gene products selected from the group consisting of Jasmonate acid Pathway, Solyc12g009220.2, auxin transporter-encoding genes, gibberellin, Solyc02g068680.1, Solyc01g095770.3, Abscisic acid signaling pathway genes, signal peptides (Solyc12g049170.2, Solyc12g049150.1, Solyc12g049070.1, Solyc09g075410.3, Solyc12g100110.1, Solyc12g100080.1, Solyc07g017570.2), Aromatic amino acid decarboxylase 1A (Solyc08g068680.3) and Fatty acyl-CoA reductase (Solyc06g074390.3), peroxidase gene (Solyc01g067870.3, Solyc11g007220.2, Solyc02g014300.2, Solyc02g082090.3, Solyc12g017870.2, Solyc01g009400.3, Solyc01g067860.3, Solyc05g055320.3), AP2/ERF (Solyc12g009240.1) and NAC (Solyc07g066330.3), WRKY23 (Solyc01g079260), and/or defense protein genes (Solyc07g009040.3, Solyc12g096920.1, Solyc07g009090.3, Solyc07g009030.3, Solyc07g009100.3, Solyc12g009240.1).

16. The method of claim 14 or 15, wherein the gene product is an RNA.

17. The method of claim 16, wherein the gene product is a messenger RNA (mRNA).

18. The method of claim 14 or 15, wherein the gene product is a protein.

19. The method of any one of claims 14 to 18, further comprising exposing the plant to a composition comprising zinc, copper and acid wherein the ratio of copper to zinc is between 1:2 and 1:20.
